(19) United States
(12) Patent Application Publication
(10) Pub. No.: US 2026/0367091 A1
(43) Pub. Date: Nov. 19, 2026

(54) Systems and Methods for Analyzing Peptide Mixtures Using Mass Mobility Tags and Mass Mobility Tagging Reagent Sets Therefor

(71) Applicant: MOBILion Systems, Inc., Chadds Ford, PA (US)

(72) Inventors: Joshua Kelly McBee, Thornton, PA (US); John Daniel DeBord, West Grove, PA (US)

(73) Assignee: MOBILion Systems, Inc., Chadds Ford, PA (US)

(21) Appl. No.: 19/680,590

(22) Filed: May 18, 2026

Related U.S. Application Data
(60) Provisional application No. 63/808,087, filed on May 19, 2025.

(51) Int. Cl. G01N 33/68; G01N 1/28

(52) U.S. Cl. CPC G01N 33/6848; G01N 1/28

(57) ABSTRACT

A peptide mixture analysis system includes a sample preparation and tagging system, ionization source, and ion mobility system. The sample preparation and tagging system prepares a plurality of peptide mixture samples, forms a plurality of mass mobility tag (MMT)-tagged peptide mixture samples, and combines the MMT-tagged peptide mixture samples into a pooled sample. The MMT-tagged peptide mixture samples are formed by reacting each peptide mixture sample with a unique MMT reagent selected from a plurality of MMT reagents, each of which includes a unique cleavable reporter tag, such that each MMT reagent changes a mobility characteristic of the peptides contained in the peptide mixture sample with which it is reacted. The ionization source ionizes the pooled sample and generates MMT-tagged peptide ions, which are received by the ion mobility system and separated thereby, such that MMT-tagged peptide ions having the same mass-to-charge ratio are separated based on ion mobility.

### CROSS-REFERENCE TO RELATED APPLICATIONS

This patent application claims priority benefit to a U.S. provisional patent application entitled “Systems and Method for Analyzing Peptide Mixtures Using Mass Mobility Tags and Mass Mobility Tagging Reagent Sets Therefor,” when was filed on May 19, 2025, and assigned Serial No. 63/808,087. The entire content of the foregoing U.S. provisional patent application is incorporated herein by reference.

### TECHNICAL FIELD

The present disclosure relates generally to the fields of proteomics, ion mobility spectrometry (IMS), and mass spectrometry (MS). More specifically, the present disclosure relates to systems and methods for multiplexed analysis of peptide mixtures, e.g., performing relative quantitation in proteomics, using mass mobility tags (MMT), ion mobility (IM) systems, and mass spectrometers, as well as mass mobility tagging reagent sets therefor.

### RELATED ART

Various technologies known in the art have been used for performing relative quantitation in proteomics, e.g., mass spectrometric analysis of proteolytic digests of protein. One such technology is isotope coded affinity tags (ICAT), which are chemical reagents that are used to label proteins for relative quantification and identification. In particular, isotope coded affinity tags are used to label proteins/peptides contained within samples, and those labeled proteins/peptides are then analyzed using liquid chromatography-mass spectrometry (LC-MS).

Another technology known in the art is tandem mass tagging (TMT), which is built off of ICAT. TMT reagents generally include three components: a primary-amine reactive component that covalently binds the reagent to a peptide, a mass-normalizing group linked to the primary-amine reactive component, and a reporter group (e.g., tag). The mass-normalizing group is isobarically labelled and linked to the reporter group by a cleavable linker. Stable isotopes, such as, ¹³C or ¹⁵N, are selectively bonded to the mass-normalizing group and the reporter group to mass balance the mass-normalizing group and the reporter group, and to cause all TMT tagged peptides to have the same mass-to-charge ratio (m/z). In this regard, the TMT reagents with various reporter masses are reacted with peptides contained within a number of different samples, and those samples are then combined, ionized, and fragmented, which causes the cleavable linker to be cleaved and a tag to be created from the cleaved reporter group. During this process, all TMT tagged peptides of a given type are simultaneously isolated and fragmented. Notably, the m/z value of the created tag is linked to the sample with which the underlying TMT reagent was reacted. The cleaved reporter groups are then detected. Depending on the number of samples and tandem mass tags used, multiple tags can be resolved in the low-mass m/z region with the intensity of each detected tag representing the relative abundance of the peptide linked to that tag from each sample.

TMT technology has been used with, for example, Orbitrap MS systems at least because such systems provide high mass resolution capable of resolving the tandem mass tags, many of which can only be resolved in the low-mass region. In contrast, other MS systems, such as quadrupole time-of-flight (QTOF) mass spectrometers, do not have sufficient mass resolution to isolate tandem mass tags that are too similar in mass.

Additionally, TMT kits are known in the art up to 32-plex, e.g., thirty-two different reagents allowing for thirty-two samples to be simultaneously analyzed (e.g., multiplexed), by utilizing a combination of ¹³C, ¹⁵N, and deuterium to mass balance the tandem mass tags.

However, TMT technology relies solely on m/z for separation and the multiplexing capacity thereof is limited.

Accordingly, there is a need for additional systems and methods for analyzing peptide mixtures that have increased multiplexing capabilities and address the foregoing problems.

### SUMMARY

The present disclosure relates to systems and methods for analyzing peptide mixtures using mass mobility tags (MMT), ion mobility (IM) systems, and mass spectrometers, as well as mass mobility tagging reagent sets therefor.

A peptide mixture analysis system includes a sample preparation and tagging system, an ionization source, and an ion mobility system. The sample preparation and tagging system prepares a plurality of peptide mixture samples, forms a plurality of mass mobility tag (MMT)-tagged peptide mixture samples, and combines the plurality of MMT-tagged peptide mixture samples into a pooled MMT-tagged peptide mixture sample. The sample preparation and tagging system forms the plurality of MMT-tagged peptide mixture samples by reacting each of the plurality of peptide mixture samples with a unique MMT reagent selected from a plurality of MMT reagents, each of which includes a unique cleavable reporter tag, such that each of the plurality of MMT reagents changes a mobility characteristic of the peptides contained in the peptide mixture sample with which the MMT reagent is reacted. The ionization source ionizes the pooled MMT-tagged peptide mixture sample and generates MMT-tagged peptide ions. The ion mobility system receives the MMT-tagged peptide ions and separates the MMT-tagged peptide ions based on ion mobility such that MMT-tagged peptide ions having the same mass-to-charge ratio are separated based on ion mobility.

In some aspects, the mobility characteristic can be collision cross section, center of mass, ion mobility, or measured arrival time.

In other aspects, each of the unique cleavable reporter tags can have a mobility characteristic that is different from the same mobility characteristic of the unique cleavable reporter tag of each of the other MMT reagents. In such aspects, the mobility characteristic can be collision cross section.

In some other aspects, the plurality of MMT reagents can include a first MMT reagent having a first unique cleavable reporter tag and a second MMT reagent having a second unique cleavable reporter tag where the first unique cleavable reporter tag has a first mobility characteristic, the second unique cleavable reporter tag has a second mobility characteristic that is different than the first mobility characteristic of the first unique cleavable reporter tag, and the first and second unique cleavable reporter tags have the same mass-to-charge ratio.

In still other aspects, each of the plurality of MMT reagents can include a mass normalizer group and an amine reactive group, and the reporter tag can be bonded to the mass normalizer group by a cleavable link.

In some aspects, the reporter tag of each of the plurality of MMT reagents can include one, two, or three heavy atoms selected from the group consisting of F, Cl, Br, I, Si, and Se.

In other aspects, each of the unique cleavable reporter tags of the plurality of MMT reagents can have the same mass-to-charge ratio, or, alternatively, each of the unique cleavable reporter tags of the plurality of MMT reagents can have a different mass-to-charge ratio.

In some other aspects, each of the unique cleavable reporter tags of the plurality of MMT reagents can include: (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

In still other aspects, the system can include a liquid chromatography system positioned downstream of the sample preparation system and upstream of the ionization source.

In other aspects, the ion mobility system can be a structures for lossless ion manipulations (SLIM) system that performs high-resolution ion mobility separations.

In some aspects, the plurality of MMT reagents can include a first group of MMT reagents and a second group of MMT reagents. In such aspects, each of the MMT reagents of the first group can have a first mass-to-charge ratio and a mobility characteristic that is different from the mobility characteristic of the other MMT reagents of the first group, while each of the MMT reagents of the second group can have a second mass-to-charge ratio that is different than the first mass-to-charge ratio and a mobility characteristic that is different from the mobility characteristic of the other MMT reagents of the second group.

In still other aspects, the plurality of MMT reagents can include a first group of MMT reagents and a second group of MMT reagents. In such aspects, each of the MMT reagents of the first group can have a first substantially similar mobility characteristic in a first range and a mass-to-charge ratio that is different than the mass-to-charge ratio of the other MMT reagents of the first group, while each of the MMT reagents of the second group can have a second substantially similar mobility characteristic in a second range that does not overlap with the first range and a mass-to-charge ratio that is different than the mass-to-charge ratio of the other MMT reagents of the second group.

In other aspects, an ion mobility spectrometry – mass spectrometry (IMS-MS) system can include the peptide mixture analysis system and a mass spectrometer, which can include a fragmentation device and a detector. In such aspects, the mass spectrometer can receive the MMT-tagged peptide ions separated based on ion mobility, fragment the MMT-tagged peptide ions so as to cleave the reporter tag from each MMT-tagged peptide ion and produce reporter ions and peptide ions, and detect the peptide ions and the reporter ions, which attribute each of the detected peptide ions to the peptide mixture sample of origination and facilitate the performance of relative quantitation of each peptide species of the plurality of peptide mixture samples.

A method of analyzing peptide mixtures includes preparing a plurality of peptide mixture samples, forming a plurality of mass mobility tag (MMT)-tagged peptide mixture samples, and combining the plurality of MMT-tagged peptide mixture samples into a pooled MMT-tagged peptide mixture sample. The plurality of MMT-tagged peptide mixture samples are formed by reacting each of the plurality of peptide mixture samples with a unique MMT reagent selected from a plurality of MMT reagents, where each of the plurality of MMT reagents includes a unique cleavable reporter tag and changes a mobility characteristic of the peptides contained in the peptide mixture sample with which the MMT reagent is reacted. The plurality of MMT-tagged peptide mixture samples are combined into a pooled MMT-tagged peptide mixture sample, which is ionized generating MMT-tagged peptide ions. The MMT-tagged peptide ions are received at an ion mobility system, which separates the MMT-tagged peptide ions based on ion mobility such that MMT-tagged peptide ions having the same mass-to-charge ratio are separated based on ion mobility.

In some aspects, the mobility characteristic can be collision cross section, center of mass, ion mobility, or measured arrival time.

In other aspects, each of the unique cleavable reporter tags can have a mobility characteristic that is different from the same mobility characteristic of the unique cleavable reporter tag of each of the other MMT reagents. In such aspects, the mobility characteristic can be collision cross section.

In some other aspects, the plurality of MMT reagents can include a first MMT reagent having a first unique cleavable reporter tag and a second MMT reagent having a second unique cleavable reporter tag where the first unique cleavable reporter tag has a first mobility characteristic, the second unique cleavable reporter tag has a second mobility characteristic that is different than the first mobility characteristic of the first unique cleavable reporter tag, and the first and second unique cleavable reporter tags have the same mass-to-charge ratio.

In still other aspects, each of the plurality of MMT reagents can include a mass normalizer group and an amine reactive group, and the reporter tag can be bonded to the mass normalizer group by a cleavable link.

In some aspects, the reporter tag of each of the plurality of MMT reagents can include one, two, or three heavy atoms selected from the group consisting of F, Cl, Br, I, Si, and Se.

In other aspects, each of the unique cleavable reporter tags of the plurality of MMT reagents can have the same mass-to-charge ratio, or, alternatively, each of the unique cleavable reporter tags of the plurality of MMT reagents can have a different mass-to-charge ratio.

In some other aspects, each of the unique cleavable reporter tags of the plurality of MMT reagents can include: (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

In still other aspects, the method can involve chromatographically separating components of the pooled MMT-tagged peptide sample with a liquid chromatography system prior to ionizing the pooled MMT-tagged peptide mixture sample.

In other aspects, the ion mobility system can be a structures for lossless ion manipulations (SLIM) system that performs high-resolution ion mobility separations.

In some aspects, the plurality of MMT reagents can include a first group of MMT reagents and a second group of MMT reagents. In such aspects, each of the MMT reagents of the first group can have a first mass-to-charge ratio and a mobility characteristic that is different from the mobility characteristic of the other MMT reagents of the first group, while each of the MMT reagents of the second group can have a second mass-to-charge ratio that is different than the first mass-to-charge ratio and a mobility characteristic that is different from the mobility characteristic of the other MMT reagents of the second group.

In still other aspects, the plurality of MMT reagents can include a first group of MMT reagents and a second group of MMT reagents. In such aspects, each of the MMT reagents of the first group can have a first substantially similar mobility characteristic in a first range and a mass-to-charge ratio that is different than the mass-to-charge ratio of the other MMT reagents of the first group, while each of the MMT reagents of the second group can have a second substantially similar mobility characteristic in a second range that does not overlap with the first range and a mass-to-charge ratio that is different than the mass-to-charge ratio of the other MMT reagents of the second group.

In other aspects, the MMT-tagged peptide ions, which have been separated based on ion mobility, can be received at a mass spectrometer including a fragmentation device and a detector, and fragmented with the fragmentation device so as to cleave the reporter tag for each MMT-tagged peptide ion and produce peptide ions and reporter ion, which attribute each of the detected peptide ions to the peptide mixture sample of origination and facilitate the performance of relative quantitation of each peptide species of the plurality of peptide mixture samples. The reporter ions and peptide ions can then be detected at the detector. In such aspects, the relative intensity of each of the reporter ions can be measured, and the relative abundance of each peptide can be compared across all samples.

A mass mobility tag (MMT) reagent set includes a plurality of unique MMT reagents that each include a unique reporter tag, a mass normalizer group, a reactive group, and a cleavable link attaching the unique reporter tag to the mass normalizer group. The unique reporter tag of each of the unique MMT reagents has a mobility characteristic that is different from the same mobility characteristic of the unique reporter tags of each of the other unique MMT reagents. Additionally, each of the unique reporter tags of the unique MMT reagents have the same mass-to-charge ratio.

In some aspects, the mobility characteristic can be collision cross section or center of mass.

In other aspects, the plurality of unique MMT reagents can each include a labeling atom that is the same for each of the plurality of unique MMT reagents, but positioned at a different labeling site of the respective unique reporter tag. In such aspects, the labeling atom can be selected from the group consisting of F, Cl, Br, I, Si, and Se.

In some other aspects, each of the unique reporter tags of the plurality of unique MMT reagents can include (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

In still other aspects, the MMT reagent set can include a second plurality of unique MMT reagents that each include a unique reporter tag, a mass normalizer group, a reactive group, and a cleavable link attaching the unique reporter tag to the mass normalizer group. The unique reporter tag of each of the unique MMT reagents of the second plurality can have a mobility characteristic that is different from the same mobility characteristic of the unique reporter tags of each of the other unique MMT reagents of the second plurality. Additionally, each of the unique reporter tags of the second plurality of unique MMT reagents can have the same mass-to-charge ratio, and the mass-to-charge ratio of the reporter tags of the second plurality of unique MMT reagents can be different than the mass-to-charge ratio of the plurality of unique MMT reagents.

A mass mobility tag (MMT) reagent set includes a plurality of unique MMT reagents that each include a unique reporter tag, a mass normalizer group, a reactive group, and a cleavable link attaching the unique reporter tag to the mass normalizer group. Each of the unique reporter tags of the unique MMT reagents have a substantially similar mobility characteristic in a first range and a mass-to-charge ratio that is different than the mass-to-charge ratio of the other unique reporter tags.

In some aspects, the mobility characteristic can be collision cross section or center of mass.

In other aspects, the plurality of unique MMT reagents can each include a labeling atom that is the same for each of the plurality of unique MMT reagents, but positioned at a different labeling site of the respective unique reporter tag. In such aspects, the labeling atom can be selected from the group consisting of F, Cl, Br, I, Si, and Se.

In some other aspects, each of the unique reporter tags of the plurality of unique MMT reagents can include (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

In still other aspects, the MMT reagent set can include a second plurality of unique MMT reagents that each include a unique reporter tag, a mass normalizer group, a reactive group, and a cleavable link attaching the unique reporter tag to the mass normalizer group. In such aspects, each of the unique reporter tags of the unique MMT reagents of the second plurality can have a substantially similar mobility characteristic in a second range that does not overlap with the first range and a mass-to-charge ratio that is different than the mass-to-charge ratio of the other unique reporter tags of the unique MMT reagents of the second plurality.

Other features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

The foregoing features of the present disclosure will be apparent from the following Detailed Description of the Invention, taken in connection with the accompanying drawings, in which:

FIG. 1 is a schematic diagram of an exemplary multiplexing peptide mixture analysis system of the present disclosure;

FIG. 2 is a diagram illustrating an exemplary mass mobility tag (MMT) reagent of the present disclosure;

FIG. 3 is hypothetical reporter tag chart of CCS value vs. m/z value for hypothetical reporter tags showing the expansion of a hypothetical 7-plex TMT reagent set into a hypothetical 21-plex TMT reagent set;

FIG. 4 is a first diagram illustrating an exemplary mass mobility tagging and analysis process according to the present disclosure;

FIG. 5 is a second diagram illustrating an exemplary mass mobility tagging and analysis process according to the present disclosure; and

FIG. 6 is a flow chart illustrating an exemplary process for analyzing a plurality of peptide mixture samples using MMT reagents and IMS.

### DETAILED DESCRIPTION

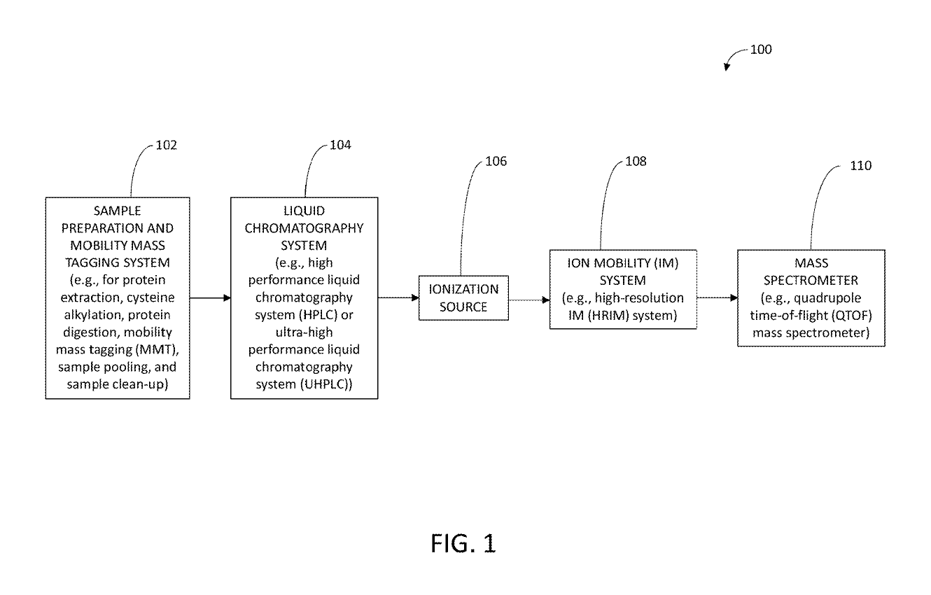

The present disclosure relates to systems and methods for analyzing peptide mixtures using mass mobility tags (MMT), ion mobility (IM) systems, and mass spectrometers, as well as mass mobility tagging reagent sets therefor, as described in detail below in connection with FIGS. 1-6.

FIG. 1 is a schematic diagram of an exemplary peptide mixture analysis system 100 in accordance with the present disclosure. The system 100 includes a sample preparation and mass mobility tagging system 102, a liquid chromatography (LC) system 104, an ionization source 106, an IM system 108, and a mass spectrometer 110.

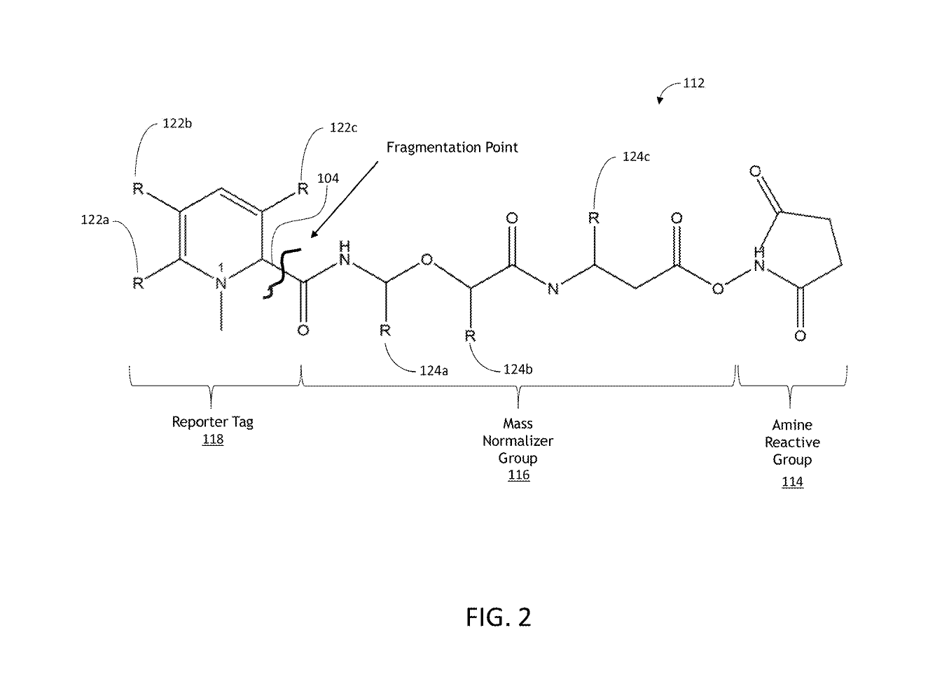

The sample preparation and mass mobility tagging system 102 prepares a plurality of samples including a peptide mixture, e.g., ninety-six samples, for analysis and reacts each sample with a mass mobility tag (MMT) reagent, such as the MMT reagents discussed in connection with FIG. 2. This sample preparation process can involve, for example, protein extraction, cysteine alkylation, and protein digestion. Once all of the samples are prepared, the sample preparation and mass mobility tagging system 102 reacts each of the prepared peptide mixture samples with a unique MMT reagent, such as the MMT reagents discussed in connection with FIG. 2, to form a plurality of MMT-tagged peptide mixture samples, which are subsequently pooled together into a pooled MMT-tagged peptide mixture sample. The sample preparation and mass mobility tagging system 102 can be an automated sample preparation system, e.g., a robotic sample preparation system, or a manual sample preparation station in which a lab technician performs the foregoing actions, e.g., via manual pipetting.

The LC system 104 can be, for example, a high-performance (or high-pressure) liquid chromatography system that receives and chromatographically separates the pooled MMT-tagged peptide mixture sample. The ionization source 106 receives the chromatographically separated sample, generates MMT-tagged peptide ions therefrom, and delivers the MMT-tagged peptide ions into the IM system 108.

The IM system 108 can be a high-resolution ion mobility (HRIM) system that is configured to separate MMT-tagged peptide ions received thereby based on ion mobility. For example, the IM system 108 can be a structure for lossless ion manipulation (SLIM) device that can systematically apply traveling and/or DC potential waveforms to the MMT-tagged peptide ions, such as the devices disclosed and described in U.S. Patent No. 8,835,839 entitled “Method and Apparatus for Ion Mobility Separations Utilizing Alternating Current Waveforms” and U.S. Patent No. 10,317,364 entitled “Ion Manipulation Device,” both of which are incorporated herein in their entirety. In this regard, the IM system 108 can include one or more surfaces, e.g., printed circuit board surfaces, that can have a plurality of electrodes arranged thereon. The electrodes can receive voltage signals, a voltage waveform, and/or a current waveform, e.g., a DC voltage or current, an RF voltage or current, or an AC voltage or current, or a superposition thereof, and can generate a potential to confine the MMT-tagged peptide ions in the IM system 108, accumulate the MMT-tagged peptide ions in the IM system 108, and guide the MMT-tagged peptide ions through the IM system 108, which can result in the accumulation and separation of the MMT-tagged peptide ions based on their mobility. It should be understood that the IM system 108 can separate the MMT-tagged peptide ions based on ion mobility using DC/traveling potential waveforms resulting from the application of one or more AC and/or DC signals, including, but not limited to, traveling wave signals, voltage gradients, voltage offsets, or combinations thereof. The IM system 108 should have resolving power capability to sufficiently separate the MMT-tagged peptide ions based on ion mobility, thus allowing the mobility dimension to be leveraged for relative quantitation.

The mass spectrometer 110 can be, for example, a quadrupole time-of-flight (QTOF) mass spectrometer. The mass spectrometer 110 receives the ion mobility separated MMT-tagged peptide ions from the IM system 108, fragments the MMT-tagged peptide ions to generate MMT reporter ions and fragmented peptide ions, and detects the MMT reporter ions and fragmented peptide ions.

It should be understood that the system 100 can be provided as a single integrated system, individual components, or a combination thereof. For example, the sample preparation and mass mobility tagging system 102 and/or the LC system 104 can be provided as separate “offline” components. As another example, the ionization source 106 and IM system 108 can be provided as a single integrated system while the MS 110 can be provided as a separate component. In this configuration, the ionization source 106 and IM system 108 combination can be integrated with different mass spectrometers 110 depending on availability and a user’s needs.

FIG. 2 is a diagram illustrating an exemplary MMT reagent 112 of the present disclosure, which is used to change a mobility characteristic of a peptide to which the MMT reagent 112 is bonded. Some exemplary mobility characteristics of a peptide that can be impacted by the MMT reagent 112 include, but are not limited to, collision cross section, center of mass, chiral centers, conformations, ion mobility (K), or any other mobility characteristic that impacts the measured ion mobility axis or measured arrival time of the peptide when analyzed with the IM system 108.

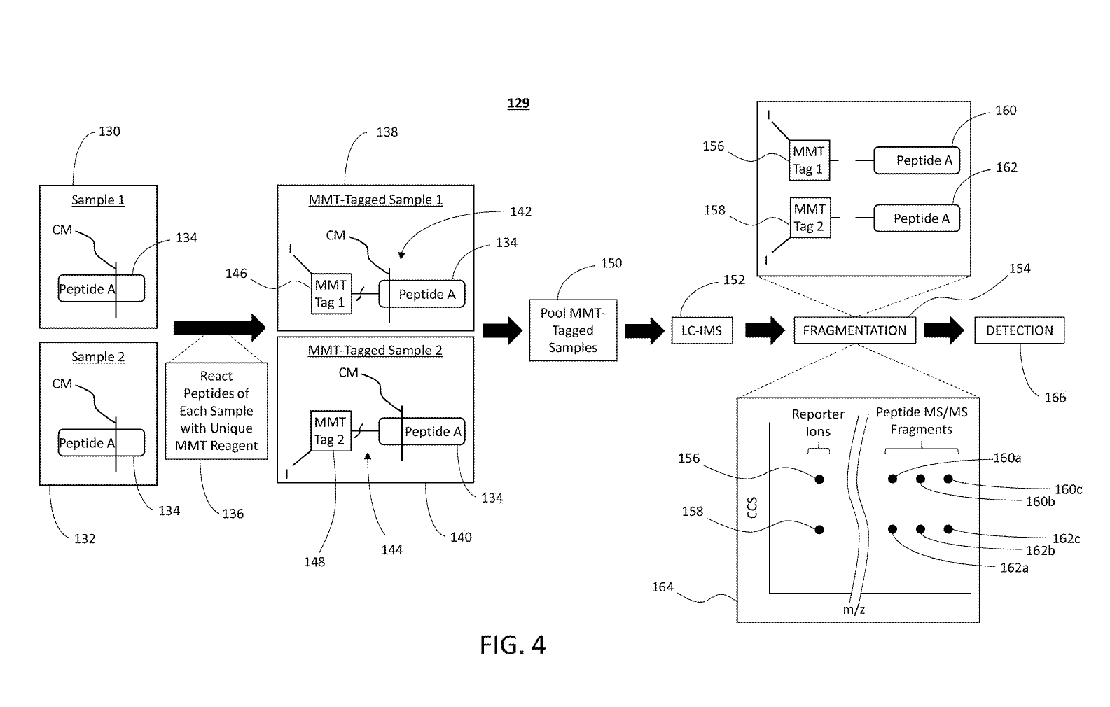

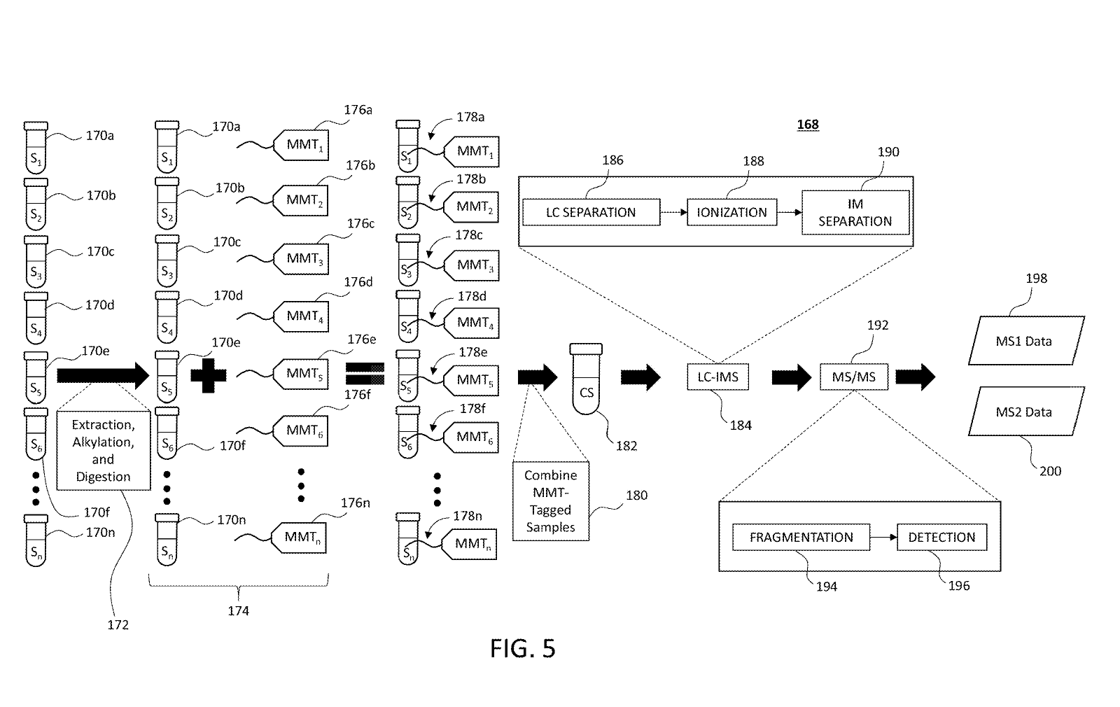

The MMT reagent 112 generally includes three linked groups, namely, a reactive group 114 (e.g., an amine or peptide reactive group), a mass normalizer group 116, and a reporter tag 118. The reactive group 114 reacts with the N-terminus amine of a peptide, and forms a covalent bond therewith, in order to bind the MMT reagent 112 to the peptide and form an MMT-tagged peptide, as discussed in greater detail in connection with FIGS. 4 and 5. The mass normalizer group 116 is linked with the reactive group 114 and to the reporter tag 118. In this regard, the mass normalizer group 116 is linked to the reporter tag 118 via a cleavable link 120 allowing the reporter tag 118 to be separated therefrom when fragmented by the MS 110, e.g., via collision-induced dissociation (CID). Generally, the MMT reagent 112 is an isomer of some form, including, but not limited to structural isomers, stereoisomers, conformational isomers, enantiomers, constitutional isomers, diasateriomers, positional isomers, geometric isomers, tautomers, etc.

The reporter tag 118 includes a plurality of reporter labeling sites 122a-c, e.g., one, two, three, four, five, etc., that can have a heavy atom bonded thereto in order to create a unique MMT reagent 112 that will shift a mobility characteristic, e.g., collision cross section, center of mass, ion mobility, etc., of a peptide when bonded thereto. A heavy atom, as it should be understood in the context of the present disclosure, is any atom having a mass greater than oxygen. Exemplary atoms that can be bonded to the reporter labeling sites 122a-c include, but are not limited to, halogen atoms such as fluorine (F), chlorine (Cl), bromine (Br), and iodine (I), halides, silicon (Si), selenium (Se), and potentially heavy metal atoms having a compatible oxidation state. Notably, atoms can be selectively bonded to the reporter labeling sites 122a-c in order to create a set of unique MMT reagents 112 that each have their own mobility characteristics and will thus differently impact, e.g., shift, the mobility characteristic(s) of the peptide to which each is bonded. For example, a reporter tag 118 having an iodine atom bonded to the first reporter labeling site 122a will have a different collision cross section than a reporter tag 118 having an iodine atom bonded to the second reporter labeling site 122b or the third reporter labeling site 122c, and will thus have a different effect on the mobility characteristics, e.g., collision cross section, center of mass, ion mobility, arrival time, etc., of the peptide to which it is bonded. That is to say, a peptide that is bonded with an MMT reagent 112 (e.g., a peptide/MMT reagent combination or MMT labelled peptide) will have different mobility characteristics than that of the peptide alone. For example, the MMT reagent 112 can shift the CCS or center of mass of the peptide (such that the peptide/MMT reagent combination has a different CCS or center of mass than the peptide alone), or change the ion mobility or arrival time of the peptide (such that the peptide/MMT reagent combination has a different arrival time than the peptide alone). Accordingly, strategically positioning atoms in the reporter labeling sites 122a-c creates reporter tags 118 and MMT reagents 112 that have different unique mobility characteristics. This is particularly advantageous as it allows for multiple reporter tags 118 to have the same m/z value, but different mobility characteristics allowing the reporter tags 118 to be separated in the mobility dimension when analyzed by the IM system 108.

Additionally and/or alternatively, bonding of the MMT reagents 112 with the respective peptides can be varied, e.g., the position of the bond can be varied, in order to selectively alter the 3-dimensional structure of the peptides and cause a similar shift in one or more mobility characteristics that impacts the measured ion mobility axis or measured arrival time of the peptide. That is, the position of the heavy atom in the MMT reagent 112 need not be varied, but instead (or in addition) bonding of the MMT reagents 112 with the respective peptides can be varied. In such instances, the shift in the one or more mobility characteristics is a result of a combination of heavy atom placement in the MMT reagent and molecular structure. Accordingly, in some instances, the same MMT reagent 112 can be used to create different shifts in mobility characteristics by varying the bonding and thus causing different changes to the 3-dimensional structure, which further expands the foregoing multiplexing capabilities.

The foregoing multiplexing capabilities can be further expanded by bonding one or more stable isotopes, such as ¹³C or ¹⁵N, to the reporter tag 118 in order to adjust the mass-to-charge ratio thereof and create additional unique reporter tags 118 and MMT reagents 112. For example, a first set of three unique reporter tags 118 can be formed by bonding zero stable isotopes to the reporter tag 118 and including an iodine atom in only the first reporter labeling site 122a for the first unique reporter tag 118, an iodine atom in only the second reporter labeling site 122b for the second unique reporter tag 118, and an iodine atom in only the third reporter labeling site 122c for the third unique reporter tag 118. Each of the reporter tags 118 of the foregoing first set have the same m/z value, but different mobility characteristics and can therefore be separated in the mobility dimension by the IM system 108. A second set of three unique reporter tags 118 can be created by taking the three unique reporter tags 118 of the first set and bonding a single ¹³C atom thereto to create a second set of three unique reporter tags 118 having the same m/z value, which is different than the m/z value of the reporter tags 118 of the first set. Additionally, the three reporter tags 118 of the second set will have the same or substantially similar mobility characteristics as the reporter tags 118 of the first set depending on the labeling site 122a-c placement of the iodine atom. Accordingly, the three reporter tags 118 of the second set will be separated from each other based on CCS, e.g., in the mobility dimension, and separated from the three reporter tags 118 of the first set based on m/z value, e.g., in the m/z dimension. That is, the reporter tags 118 allow for two-dimensional separation. Thus, the 3-plex MMT reagent 112 set (e.g., the first reagent set) can be expanded into a 6-plex MMT reagent 112 set by adding a single stable isotope to the reporter tags 118.

The foregoing first and second MMT reagent 112 sets can be further expanded by adding a second stable isotope to the report tag 118 to form a third set (forming a 9-plex MMT reagent 112 set), a third stable isotope to the report tag 118 to form a fourth set (forming a 12-plex MMT reagent 112 set), a fourth stable isotope to the report tag 118 to form a fifth set (forming a 15-plex MMT reagent 112 set), a fifth stable isotope to the report tag 118 to form a sixth set (forming an 18-plex MMT reagent 112 set), a sixth stable isotope to the report tag 118 to form a seventh set (forming a 21-plex MMT reagent 112 set), etc., as shown, for example, in FIG. 4, which is discussed in greater detail below. While the foregoing discussion only references up to six stable isotopes being added, it should be understood that additional stable isotopes could be added to further expand the MMT reagent sets. For example, sixteen stable isotopes could be added which would allow for up to 16-plex in the m/z dimension, e.g., MMT reagents 112 having sixteen unique m/z values.

Additionally, the foregoing MMT reagent 112 sets can be even further expanded by bonding additional atoms to the reporter labeling sites 122a-c, e.g., by providing an iodine atom in (a) the first and second reporter labeling sites 122a, 122b, (b) the second and third reporter labeling sites 122b, 122c, or (c) the first, second, and third reporter labeling sites 122a-c, or by using a reporter tag 118 that includes more than three, e.g., six, different reporter labeling sites 122a-c. In such instances, the expanded chemistry allows for at least 6-plex in the mobility dimension, e.g., six unique mobility characteristics (such as collision cross section, ion mobility, or center of mass). Combining the potential 6-plex in the mobility dimension with the potential 16-plex in the m/z dimension allows for the mass mobility tagging operation of the present disclosure to be expanded to 96-plex total. This is particularly beneficial as it allows for more samples to be analyzed in a single experiment, and for a 96-well plate to be used for sample handling and preparation, which is readily compatible with liquid handling automation systems, thus adding reliability and accuracy to an experiment.

It should be understood that the foregoing discussed unique MMT reagents 112 can be provided as sets of multiple MMT reagents 112. For example, each set of MMT reagents 112 can have the same m/z value, but different mobility characteristics. Alternatively, each set of MMT reagents 112 can have the same or substantially similar mobility characteristics (e.g., within a range that the IM system 108 and mass spectrometer 110 do not differentiate or otherwise measure a significant difference within, such as, for example, +/- ~2.5% CCS or +/- ~0.5% CCS for high resolution ion mobility systems), but different m/z values.

In addition to the foregoing, the mass normalizer group 116 of each MMT reagent 112 includes a plurality of labeling sites 124a-c that can each have an atom, e.g., a stable isotope such as ¹³C or ¹⁵N, selectively bonded thereto in order to mass balance the MMT reagent 112. In this regard, the mass normalizer group 116 can be specifically designed to have a mass that balances the mass of the reporter tag 118. It is noted that the mass normalizer group 116 can have more or less labeling sites 124a-c than those shown in FIG. 2.

In some aspects, the MMT reagents 112 can be designed so as to not have a measurable impact on the chromatographic separation performed by the liquid chromatography system 104, which can result in increased utility. That is, the MMT reagents can be formulated and designed such that they do not cause chromatographic retention time shifts of the peptide when bonded thereto (e.g., the MMT labelled peptide and the original peptide will have the same or substantially similar chromatographic retention times).

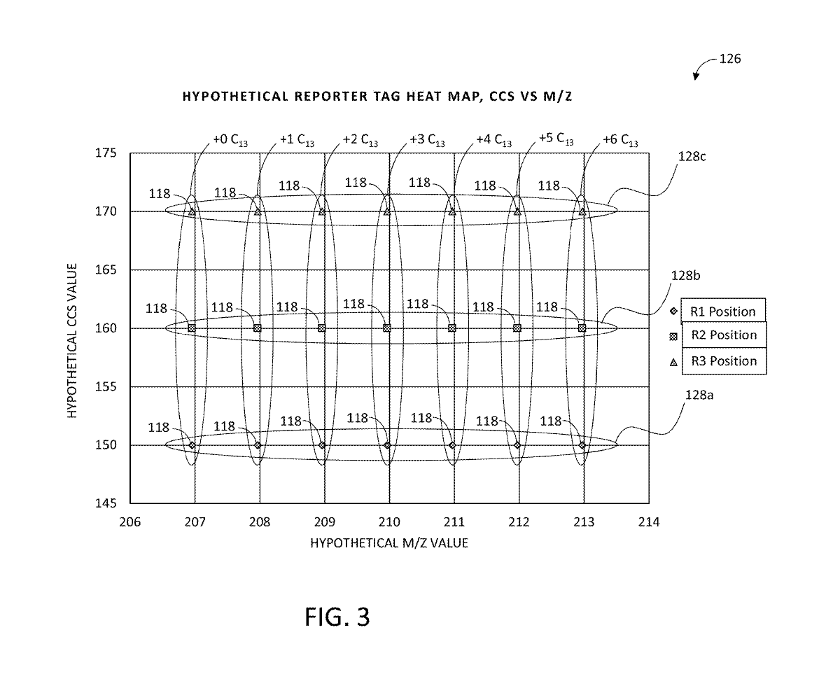

FIG. 3 is hypothetical reporter tag chart 126 of CCS value vs. m/z value for hypothetical reporter tags showing the expansion of a hypothetical 7-plex TMT reagent set into a hypothetical 21-plex TMT reagent set. It is noted that the CCS values provided in the chart 126 are hypothetical in nature and more generally provide a representation of mobility separation due to positional placement of a heavy atom in one of the reporter labeling sites 122a-c of the reporter tag 118, as described below.

The chart 126 illustrates a first set of reporter tags 128a, a second set of reporter tags 128b, and a third set of reporter tags 128c. The first set of reporter tags 128a includes seven reporter tags 118 that each have an iodine atom in the first reporter labeling site 122a with a varying number of C₁₃ atoms bonded to the reporter tag 118, e.g., 0, 1, 2, 3, 4, 5, or 6 C₁₃ atoms. Accordingly, each of the reporter tags 118 in the first set 128a have a substantially similar CCS value, e.g., a CCS value that is within a first range (which can be, for example, a range that the IM system 108 and mass spectrometer 110 do not differentiate or otherwise measure a significant difference within, such as, for example, +/- ~2.5% CCS or +/- ~0.5% CCS for high resolution ion mobility systems), but different m/z values due to the different number of C₁₃ atoms bonded thereto.

The second set of reporter tags 128b includes seven reporter tags 118 that each have an iodine atom in the second reporter labeling site 122b with a varying number of C₁₃ atoms bonded to the reporter tag 118, e.g., 0, 1, 2, 3, 4, 5, or 6 C₁₃ atoms. Accordingly, each of the reporter tags 118 in the second set 128b have a substantially similar CCS value, e.g., a CCS value that is within a second range (which can be, for example, a range that the IM system 108 and mass spectrometer 110 do not differentiate or otherwise measure a significant difference within, such as, for example, +/- ~2.5% CCS or +/- ~0.5% CCS for high resolution ion mobility systems), but different m/z values due to the different number of C₁₃ atoms bonded thereto. Additionally, as can be seen in FIG. 3, the reporter tags 118 in the second set 128b have the same m/z values as the vertically adjacent reporter tags 118 of the first set 128a based on the number of C₁₃ atoms bonded thereto, but different (increased) CCS values due to the placement of the iodine atom in the second reporter labeling site 122b (e.g., the second range of CCS values does not overlap the first range of CCS values).

The third set of reporter tags 128c includes seven reporter tags 118 that each have an iodine atom in the third reporter labeling site 122c with a varying number of C₁₃ atoms bonded to the reporter tag 118, e.g., 0, 1, 2, 3, 4, 5, or 6 C₁₃ atoms. Accordingly, each of the reporter tags 118 in the third set 128c have a substantially similar CCS value, e.g., a CCS value that is within a third range (which can be, for example, a range that the IM system 108 and mass spectrometer 110 do not differentiate or otherwise measure a significant difference within, such as, for example, +/- ~2.5% CCS or +/- ~0.5% CCS for high resolution ion mobility systems), but different m/z values due to the different number of C₁₃ atoms bonded thereto. Additionally, as can be seen in FIG. 3, the reporter tags 118 in the third set 128c have the same m/z values as the vertically adjacent reporter tags 118 of the first and second sets 128a, 128b based on the number of C₁₃ atoms bonded thereto, but different (increased) CCS values due to the placement of the iodine atom in the third reporter labeling site 122c (e.g., the third range of CCS values does not overlap the first range of CCS values or the second range of CCS values).

Accordingly, FIG. 3 demonstrates that a hypothetical 7-plex MMT reagent set where the reporter tag 118 includes between 0 and 6 C₁₃ atoms can be expanded into a 21-plex MMT reagent set by strategically positioning a heavy atom in the reporter tag 118. Furthermore, expanded chemistry would allow for the exemplary reporter tags of FIG. 3 to be increased to 6-plex in the mobility dimension, e.g., six unique CCS values, and 16-plex in the m/z dimension, e.g., sixteen unique m/z values. Combining the potential 6-plex in the mobility dimension with the potential 16-plex in the m/z dimension allows for 96-plex total. That is, the MMT technique of the present disclosure can be expanded to provide 96 unique MMT reagents that can be simultaneously used in a single experiment.

FIG. 4 is a diagram illustrating an exemplary mass mobility tagging and analysis process 129. A first peptide mixture sample 130 and a second peptide mixture sample 132 are provided that each contain Peptide A 134, which has a center of mass CM. In operation 136, each of the peptide mixture samples 130, 132 is reacted with a unique MMT reagent 112 (see FIG. 2) to form MMT-tagged peptide mixture samples 138, 140, each of which contain MMT-tagged peptides 142, 144 that are formed from Peptide A 134 bonded with the respective unique MMT reagent 112, e.g., via the N-terminal amine of Peptide A 134 and the reactive group 114 of the MMT reagents 112. Accordingly, each of the MMT-tagged peptides 142, 144 include a unique MMT tag 146, 148, which is based on the unique MMT reagent 112 reacted with the respective sample 130, 132. As can be seen in FIG. 4, each unique MMT tag 146, 148 includes an iodine atom in a different labeling location, which results in the unique MMT tags 146, 148 having different mobility characteristics. Since the unique MMT tags 146, 148 have different mobility characteristics, they in turn each impact the mobility characteristics of Peptide A 134 differently. In particular, as shown in FIG. 4, the first MMT tag 146 shifts the center of mass CM of Peptide A 134 further than the second MMT tag 148 shifts the center of mass CM of Peptide A 134. Accordingly, the MMT-tagged peptides 142, 144 will have different mobility characteristics, but the same m/z value, such that they can be separated by mobility when analyzed by the IM system 108.

In operation 150, the MMT-tagged peptide mixture samples 138, 140 are pooled together, e.g., combined, to form a pooled MMT-tagged peptide mixture sample that includes both MMT-tagged peptides 142, 144. The pooled MMT-tagged peptide mixture sample then undergoes LC-IMS 152 using the LC system 104, which can be on-line or off-line, ionization source 106, and IM system 108 of FIG. 1. In this regard, the MMT-tagged peptides 142, 144 of the pooled MMT-tagged peptide mixture sample are ionized by the ionization source 106 to generate MMT-tagged peptide ions, which are separated based on ion mobility by the IM system 108 made possible by the unique MMT-tags 146, 148.

The mobility separated MMT-tagged peptide ions then enter the mass spectrometer 110 and are fragmented in operation 154, which can be performed via collision-inducted dissociation (CID). In particular, fragmentation causes the MMT-tags 146, 148 to be cleaved at the cleavable link 120 (see FIG. 2) resulting in the formation of reporter ions 156, 158, which respectively correspond to the MMT-tags 146, 148 (e.g., reporter tag 118 from FIG. 2), and peptide ions 160, 162, which can also be fragmented into peptide fragments. Chart 164 is a hypothetical plot of CCS vs. m/z for hypothetical reporter ions 156, 158 and peptide fragments 160a-c, 162a-c generated by the fragmentation of the MMT-tagged peptide ions. As can be seen in chart 164, each of the reporter ions 156, 158 have a specific m/z value and CCS, which are different due to the positioning of the iodine atom in the MMT-tags 146, 148. Additionally, the reporter ions 156, 158 are generally at a lower m/z value than the peptide fragments 160a-c, 162a-c. The reporter ions 156, 158 and the peptide fragments 160a-c, 162a-c are then detected by the mass spectrometer 110 in operation 166. In this regard, the mass spectrometer 110 acquires a mass spectrum of the reporter ions 156, 158 and peptide fragments 160a-c, 162a-c such that the abundances of reporter ions 156, 158 and peptide fragments 160a-c, 162a-c at specific CCS values in the mobility dimension phase and at specific m/z values are determined by the mass spectrometer 110.

FIG. 5 is a second diagram illustrating an exemplary mass mobility tagging and analysis process 168 according to the present disclosure. First, a plurality of samples 170a-n that each include a peptide mixture are provided. For example, the samples 170a-n could be provided to the sample preparation and mass mobility tagging system 102 for sample preparation. The samples 170a-n could be provided by an automated system or manually by a lab technician. The samples 170a-n are then prepared in operation 172 using known techniques in the art, including but not limited to, protein extraction, alkylation, and digestion. The sample preparation operation 172 can be performed by or at the sample preparation and mass mobility tagging system 102 either manually or autonomously.

Next, an MMT-tagging operation 174 is performed in which each of the samples 170a-n is reacted with a unique MMT reagent 176a-n to form MMT-tagged peptide mixture samples 178a-n. During the MMT-tagging operation 174, each of the unique MMT reagents 176a-n binds to the peptides contained within the respective sample 170a-n, which results in a change to or shifting of mobility characteristics of the peptides. Each unique MMT reagent 176a-n can have a unique mobility characteristic (e.g., CCS) and m/z value combination, as discussed in connection with FIGS. 2 and 3. Accordingly, each of the unique MMT reagents 176a-n, and the reporter tag 118 thereof, identify the sample to which it is reacted, which is of particular utility during MS-MS analysis. The MMT-tagged peptide mixture samples 178a-n are then combined in operation 180 into a pooled MMT-tagged peptide mixture sample 182. It is noted that operations 172, 174, and 180 can be performed online or offline. Additionally, operations 172, 174, and 180 can be performed autonomously by an autonomous fluid handling system or manually by a lab technician.

The pooled MMT-tagged peptide mixture sample 182 then undergoes LC-IMS 184, which can be performed by the LC system 104, ionization source 106, and IM system 108 shown and described in connection with FIG. 1. In this regard, the LC system 104 can be an offline system that is not integrated with the ionization source 106 or the IM system 108. During LC-IMS 184, the pooled MMT-tagged peptide mixture sample 182 first undergoes chromatographic separation 186 and then ionization 188 to form MMT-tagged peptide ions that are injected into the IM system 108 and undergo IM separation 190 thereby. In this regard, the IM system 108 separates the MMT-tagged peptide ions based on ion mobility, which, as discussed above in connection with FIG. 2, is directly impacted by the unique MMT reagent 176a-n bound to the peptides.

The mobility separated MMT-tagged peptide ions then undergo MS/MS analysis 192 by the mass spectrometer 110. During the MS/MS analysis 192, the mobility separated MMT-tagged peptide ions can undergo fragmentation 194 in which the MMT-tags 118 are cleaved at the cleavable link 120 (see FIG. 2) resulting in the formation of reporter ions, which correspond to the reporter tags 118 of the unique MMT reagents 176a-n, and peptide ions, which can also be fragmented into peptide fragments. The reporter ions and peptide fragments are then detected by the mass spectrometer 110 in operation 196. MS1 data 198 and MS2 data 200 is then generated from the detected reporter ions and peptide fragments.

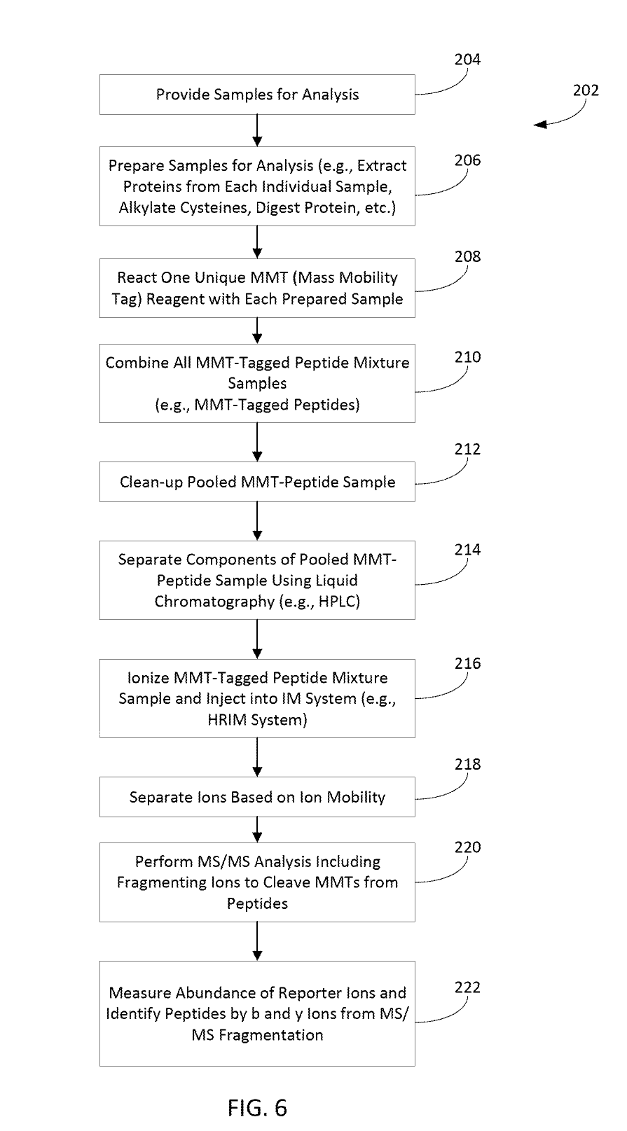

FIG. 6 is a flow chart 202 illustrating an exemplary process for analyzing a plurality of peptide mixture samples using MMT reagents and IMS. In step 204, a plurality of biological samples each containing a peptide mixture are provided for analysis to the sample preparation and mass mobility tagging system 102. The plurality of biological samples can be provided to the sample preparation and mass mobility tagging system 102 by a lab technician, e.g., manually, or by a robotic fluid handling system, e.g., autonomously. It is noted that the sample preparation and mass mobility tagging system 102 can be an online or offline system or station.

In step 206, each sample is prepared for analysis by the sample preparation and mass mobility tagging system 102. The sample preparation of step 206 can include, but is not limited to, protein extraction, cysteine alkylation, protein digestion, etc. In step 208, each of the prepared samples is reacted with a unique MMT reagent 112, such as those described in connection with FIGS. 2 and 3, to form MMT-tagged peptide mixture samples. Reacting each of the prepared samples with a unique MMT reagent 112 causes each of the peptides contained within the prepared sample to bond with the unique MMT reagent 112, which changes a mobility characteristic of the peptide allowing the peptide/MMT reagent combinations from all samples to be separated based on mobility by the IM system 108. That is, the peptide/MMT reagent combinations will have different mobility characteristics than just the peptide of that combination. For example, the MMT reagent 112 can shift the CCS or center of mass of the peptide (such that the peptide/MMT reagent combination has a different CCS or center of mass than the peptide alone), or change the ion mobility or the arrival time of the peptide (such that the peptide/MMT reagent combination has a different measured arrival time than what would be expected for the peptide alone). In step 210, all of the MMT-tagged peptide mixture samples are combined into a single pooled MMT-tagged peptide mixture sample. Steps 206, 208, and 210 can be performed by or at the sample preparation and mass mobility tagging system 102. In this regard, step 206, 208, and 210 can be performed manually by a lab technician or autonomously by a robotic fluid handling system.

In step 212, the pooled MMT-tagged peptide mixture sample is cleaned-up, e.g., using solid phase extraction or similar operation, prior to LC. In step 214, the components of the pool MMT-peptide sample are separated via liquid chromatography (e.g., HPLC), which can be performed by the LC system 104. It is noted that the LC system 104 can be an offline or online system. In step 216, the MMT-tagged peptide mixture sample is ionized using the ionization source 106 forming MMT-tagged peptide ions that are injected into the IM system 108, which can be a SLIM system capable of performing high-resolution ion mobility separations. In this regard, the IM system 108 should have sufficient resolving power to separate the MMT-tagged peptide ions based on ion mobility. In step 218, the MMT-tagged peptide ions are separated based on ion mobility by the IM system 108. Next, in step 220, the MMT-tagged peptide ions that have been separated based on ion mobility undergo MS/MS analysis by the mass spectrometer 110, which can be a QTOF. During the MS/MS analysis, the MMT-tagged peptide ions can be fragmented, which can occur within the mass spectrometer 110, causing the reporter tags 118 to be cleaved at the cleavable link 120 (see FIG. 2) and resulting in the formation of reporter ions, which correspond to the reporter tags 118 of the unique MMT reagents 112. Additionally, during fragmentation, the peptide ions can be fragmented into peptide fragments. The reporter ions and peptide fragments are detected by the mass spectrometer 110 during the MS/MS analysis of step 220. In step 222, the abundance of reporter ions is measured and peptides are identified by detected b and y ions from the MS/MS fragmentation. MS1 data and MS2 data can be generated from the MS/MS analysis.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure. The features illustrated or described in connection with one exemplary embodiment may be combined with the features of other embodiments. Such modifications and variations are intended to be included within the scope of the present disclosure. Further, in the present disclosure, like-named components of the embodiments generally have similar features, and thus within a particular embodiment each feature of each like-named component is not necessarily fully elaborated upon.

What is claimed is:

1. A peptide mixture analysis system, comprising:
a sample preparation and tagging system, the sample preparation and tagging system:
preparing a plurality of peptide mixture samples,
forming a plurality of mass mobility tag (MMT)-tagged peptide mixture samples by reacting each of the plurality of peptide mixture samples with a unique MMT reagent selected from a plurality of MMT reagents, each of the plurality of MMT reagents changing a mobility characteristic of the peptides contained in the peptide mixture sample with which the MMT reagent is reacted, and
combining the plurality of MMT-tagged peptide mixture samples into a pooled MMT-tagged peptide mixture sample;
an ionization source, the ionization source ionizing the pooled MMT-tagged peptide mixture sample and generating MMT-tagged peptide ions; and
an ion mobility system, the ion mobility system receiving the MMT-tagged peptide ions and separating the MMT-tagged peptide ions based on ion mobility such that MMT-tagged peptide ions having the same mass-to-charge ratio are separated based on ion mobility,
wherein each of the plurality of MMT reagents includes a unique cleavable reporter tag.

2. The system of claim 1, wherein the mobility characteristic is collision cross section, center of mass, ion mobility, or measured arrival time.

3. The system of claim 1, wherein each of the unique cleavable reporter tags has a mobility characteristic that is different from the same mobility characteristic of the unique cleavable reporter tag of each of the other MMT reagents.

4. The system of claim 3, wherein the mobility characteristic is collision cross section.

5. The system of claim 1, wherein (i) the plurality of MMT reagents includes a first MMT reagent having a first unique cleavable reporter tag and a second MMT reagent having a second unique cleavable reporter tag, (ii) the first unique cleavable reporter tag has a first mobility characteristic, (iii) the second unique cleavable reporter tag has a second mobility characteristic that is different than the first mobility characteristic of the first unique cleavable reporter tag, and (iv) the first and second unique cleavable reporter tags have the same mass-to-charge ratio.

6. The system of claim 1, wherein each of the plurality of MMT reagents includes a mass normalizer group and an amine reactive group, the reporter tag being bonded to the mass normalizer group by a cleavable link.

7. The system of claim 1, wherein the reporter tag of each of the plurality of MMT reagents includes one, two, or three heavy atoms selected from the group consisting of F, Cl, Br, I, Si, and Se.

8. The system of claim 1, wherein each of the unique cleavable reporter tags of the plurality of MMT reagents has the same mass-to-charge ratio.

9. The system of claim 1, wherein each of the unique cleavable reporter tags of the plurality of MMT reagents has a different mass-to-charge ratio.

10. The system of claim 1, wherein each of the unique cleavable reporter tags of the plurality of MMT reagents includes (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

11. The system of claim 1, comprising a liquid chromatography system positioned downstream of the sample preparation system and upstream of the ionization source.

12. The system of claim 1, wherein the ion mobility system is a structures for lossless ion manipulations (SLIM) system that performs high-resolution ion mobility separations.

13. The system of claim 1, wherein:
the plurality of MMT reagents includes a first group of MMT reagents and a second group of MMT reagents,
each of the MMT reagents of the first group have a first mass-to-charge ratio and a mobility characteristic that is different from the mobility characteristic of the other MMT reagents of the first group,
each of the MMT reagents of the second group have a second mass-to-charge ratio and a mobility characteristic that is different from the mobility characteristic of the other MMT reagents of the second group, and
the second mass-to-charge ratio is different than the first mass-to-charge ratio.

14. The system of claim 1, wherein:
the plurality of MMT reagents includes a first group of MMT reagents and a second group of MMT reagents,
each of the MMT reagents of the first group have a first substantially similar mobility characteristic in a first range and a mass-to-charge ratio that is different than the mass-to-charge ratio of the other MMT reagents of the first group,
each of the MMT reagents of the second group have a second substantially similar mobility characteristic in a second range and a mass-to-charge ratio that is different than the mass-to-charge ratio of the other MMT reagents of the second group, and
the second range does not overlap with the first range.

15. An ion mobility spectrometry – mass spectrometry (IMS-MS) system, comprising:
the peptide mixture analysis system of claim 1; and
a mass spectrometer including a fragmentation device and a detector, the mass spectrometer:
receiving the MMT-tagged peptide ions separated based on ion mobility,
fragmenting the MMT-tagged peptide ions so as to cleave the reporter tag from each MMT-tagged peptide ion, and produce reporter ions and peptide ions, and
detecting the reporter ions and the peptide ions,
wherein the reporter ions attribute each of the detected peptide ions to the peptide mixture sample of origination and facilitate the performance of relative quantitation of each peptide species of the plurality of peptide mixture samples.

16. A method of analyzing peptide mixtures, comprising:
preparing a plurality of peptide mixture samples;
forming a plurality of mass mobility tag (MMT)-tagged peptide mixture samples by reacting each of the plurality of peptide mixture samples with a unique MMT reagent selected from a plurality of MMT reagents, each of the plurality of MMT reagents including a unique cleavable reporter tag and changing a mobility characteristic of the peptides contained in the peptide mixture sample with which the MMT reagent is reacted;
combining the plurality of MMT-tagged peptide mixture samples into a pooled MMT-tagged peptide mixture sample;
ionizing the pooled MMT-tagged peptide mixture sample and generating MMT-tagged peptide ions;
receiving the MMT-tagged peptide ions at an ion mobility system; and
separating, with the ion mobility system, the MMT-tagged peptide ions based on ion mobility such that MMT-tagged peptide ions having the same mass-to-charge ratio are separated based on ion mobility.

17. The method of claim 16, wherein the mobility characteristic is collision cross section, center of mass, ion mobility, or measured arrival time.

18. The method of claim 16, wherein each of the reporter tags has a mobility characteristic that is different from the same mobility characteristic of the reporter tag of each of the other MMT reagents.

19. The method of claim 18, wherein the mobility characteristic is collision cross section.

20. The method of claim 16, wherein (i) the plurality of MMT reagents includes a first MMT reagent having a first unique cleavable reporter tag and a second MMT reagent having a second unique cleavable reporter tag, (ii) the first unique cleavable reporter tag has a first mobility characteristic, (iii) the second unique cleavable reporter tag has a second mobility characteristic that is different than the first mobility characteristic of the first unique cleavable reporter tag, and (iv) the first and second unique cleavable reporter tags have the same mass-to-charge ratio.

21. The method of claim 16, wherein each of the plurality of MMT reagents includes a mass normalizer group and an amine reactive group, the reporter tag being bonded to the mass normalizer group by a cleavable link.

22. The method of claim 16, wherein the reporter tag of each of the plurality of MMT reagents includes one, two, or three heavy atoms selected from the group consisting of F, Cl, Br, I, Si, and Se.

23. The method of claim 16, wherein each of the unique cleavable reporter tags of the plurality of MMT reagents has the same mass-to-charge ratio.

24. The method of claim 16, wherein each of the unique cleavable reporter tags of the plurality of MMT reagents has a different mass-to-charge ratio.

25. The method of claim 16, wherein each of the unique cleavable reporter tags of the plurality of MMT reagents includes (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

26. The method of claim 16, comprising:
chromatographically separating components of the pooled MMT-tagged peptide sample with a liquid chromatography system prior to ionizing the pooled MMT-tagged peptide mixture sample.

27. The method of claim 16, wherein the ion mobility system is a structures for lossless ion manipulations (SLIM) system that performs high-resolution ion mobility separations.

28. The method of claim 16, wherein:
the plurality of MMT reagents includes a first group of MMT reagents and a second group of MMT reagents,
each of the MMT reagents of the first group have a first mass-to-charge ratio and a mobility characteristic that is different from the mobility characteristic of the other MMT reagents of the first group,
each of the MMT reagents of the second group have a second mass-to-charge ratio and a mobility characteristic that is different from the mobility characteristic of the other MMT reagents of the second group, and
the second mass-to-charge ratio is different than the first mass-to-charge ratio.

29. The method of claim 16, wherein:
the plurality of MMT reagents includes a first group of MMT reagents and a second group of MMT reagents,
each of the MMT reagents of the first group have a first substantially similar mobility characteristic in a first range and a mass-to-charge ratio that is different than the mass-to-charge ratio of the other MMT reagents of the first group,
each of the MMT reagents of the second group have a second substantially similar mobility characteristic in a second range and a mass-to-charge ratio that is different than the mass-to-charge ratio of the other MMT reagents of the second group, and
the second range does not overlap with the first range.

30. The method of claim 16, comprising:
receiving, at a mass spectrometer including a fragmentation device and a detector, the MMT-tagged peptide ions separated based on ion mobility;
fragmenting, with the fragmentation device, the MMT-tagged peptide ions so as to cleave the reporter tag for each MMT-tagged peptide ion, and produce reporter ions and peptide ions; and
detecting, at the detector, the reporter ions and the peptide ions,
wherein the reporter ions attribute each of the detected peptide ions to the peptide mixture sample of origination and facilitate the performance of relative quantitation of each peptide species of the plurality of peptide mixture samples.

31. The method of claim 30, comprising:
measuring the relative intensity of each of the reporter ions; and
comparing the relative abundance of each peptide across all samples.

32. A mass mobility tag (MMT) reagent set, comprising:
a plurality of unique MMT reagents, each of the plurality of unique MMT reagents including a unique reporter tag, a mass normalizer group, a reactive group, and a cleavable link attaching the unique reporter tag to the mass normalizer group,
wherein the unique reporter tag of each of the unique MMT reagents has a mobility characteristic that is different from the same mobility characteristic of the unique reporter tags of each of the other unique MMT reagents, and
wherein each of the unique reporter tags of the unique MMT reagents have the same mass-to-charge ratio.

33. The MMT reagent set of claim 32, wherein the mobility characteristic is collision cross section or center of mass.

34. The MMT reagent set of claim 32, wherein the plurality of unique MMT reagents each includes a labeling atom positioned at a different labeling site of the respective unique reporter tag, the labeling atom being the same for each of the plurality of unique MMT reagents.

35. The MMT reagent set of claim 34, wherein the labeling atom is selected from the group consisting of F, Cl, Br, I, Si, and Se.

36. The MMT reagent set of claim 32, wherein each of the unique reporter tags of the plurality of unique MMT reagents includes (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

37. The MMT reagent set of claim 32, comprising:
a second plurality of unique MMT reagents, each of the second plurality of unique MMT reagents including a unique reporter tag, a mass normalizer group, a reactive group, and a cleavable link attaching the unique reporter tag to the mass normalizer group,
wherein the unique reporter tag of each of the unique MMT reagents of the second plurality has a mobility characteristic that is different from the same mobility characteristic of the unique reporter tags of each of the other unique MMT reagents of the second plurality,
wherein each of the unique reporter tags of the second plurality of unique MMT reagents have the same mass-to-charge ratio, and
wherein the mass-to-charge ratio of the reporter tags of the second plurality of unique MMT reagents is different than the mass-to-charge ratio of the plurality of unique MMT reagents.

38. A mass mobility tag (MMT) reagent set, comprising:
a plurality of unique MMT reagents, each of the plurality of unique MMT reagents including a unique reporter tag, a mass normalizer group, a reactive group, and a cleavable link attaching the unique reporter tag to the mass normalizer group,
wherein each of the unique reporter tags of the unique MMT reagents have a substantially similar mobility characteristic in a first range and a mass-to-charge ratio that is different than the mass-to-charge ratio of the other unique reporter tags.

39. The MMT reagent set of claim 38, wherein the mobility characteristic is collision cross section or center of mass.

40. The MMT reagent set of claim 38, wherein the plurality of unique MMT reagents each includes a labeling atom positioned at the same labeling site of the respective unique reporter tag, the labeling atom being the same for each of the plurality of unique MMT reagents.

41. The MMT reagent set of claim 40, wherein the labeling atom is selected from the group consisting of F, Cl, Br, I, Si, and Se.

42. The MMT reagent set of claim 38, wherein each of the unique reporter tags of the plurality of unique MMT reagents includes (i) one C₁₃ atom, (ii) two C₁₃ atoms, (iii) three C₁₃ atoms, (iv) four C₁₃ atoms, (v) five C₁₃ atoms, (vi) six C₁₃ atoms, (vii) one N₁₅ atom, (viii) one C₁₃ atom and one N₁₅ atom, (ix) two C₁₃ atoms and one N₁₅ atom, (x) three C₁₃ atoms and one N₁₅ atom, (xi) four C₁₃ atoms and one N₁₅ atom, (xii) five C₁₃ atoms and one N₁₅ atom, or (xiii) six C₁₃ atoms and one N₁₅ atom.

43. The MMT reagent set of claim 38, comprising:
a second plurality of unique MMT reagents, each of the second plurality of unique MMT reagents including a unique reporter tag, a mass normalizer group, a reactive group, and a cleavable link attaching the unique reporter tag to the mass normalizer group,
wherein each of the unique reporter tags of the unique MMT reagents of the second plurality have a substantially similar mobility characteristic in a second range and a mass-to-charge ratio that is different than the mass-to-charge ratio of the other unique reporter tags of the unique MMT reagents of the second plurality, and
wherein the second range does not overlap with the first range.
